SEQUENCE LISTING (13 sequences)

SEQ ID NO: 1 - protein, 407 aa, Macaca Mulatta
  MISC_FEATURE: rh PHD1
MDSPCQSQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCGCSSGSGEASAGLMEEALPSAPERLALDYIVPCMRYYGICVKDSFLGAALGGRVLAEVEALKRGGRLRDGQLVSQRAIPPRSIRGDQIAWVEGHEAGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 2 - protein, 426 aa, Macaca Mulatta
  MISC_FEATURE: rh PHD2
MANDSGGPGGPSPSERDRQYCELCGKMENLLRCSRCRSSFYCCKEHQRQDWKKHKLVCQGSEGALGPGVGPHQHSGPAPPVAAPPPRAGAREPRKAAARRDNASGDAAKAKAKAKPPADPAAAASPSRAAPGGQGSAVAAEAEPGKEEPPARSSLFQEKANLYPPSNTPGDALSPSGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 3 - protein, 239 aa, Macaca Mulatta
  MISC_FEATURE: rh PHD3
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGNYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 4 - protein, 407 aa, Homo Sapiens
MDSPCQPQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCSCSSGSGEASAGLMEEALPSAPERLALDYIVPCMRYYGICVKDSFLGAALGGRVLAEVEALKRGGRLRDGQLVSQRAIPPRSIRGDQIAWVEGHEPGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 5 - protein, 426 aa, Homo Sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 6 - protein, 239 aa, Homo Sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 7 - protein, 19 aa, Artificial Sequence
  peptide sequence
DLDLEMLAPYIPMDDDFQL

SEQ ID NO: 8 - protein, 19 aa, Artificial Sequence
  peptide sequence
DLDLEMLAPYIGMDDDFQL

SEQ ID NO: 9 - protein, 19 aa, Artificial Sequence
  peptide sequence
DLDLEALAPYIPADDDFQL

SEQ ID NO: 10 - protein, 19 aa, Artificial Sequence
  peptide sequence
DLDLEALAPYIGADDDFQL

SEQ ID NO: 11 - protein, 5 aa, Artificial Sequence
  peptide sequence
LAPYI

SEQ ID NO: 12 - protein, 6 aa, Artificial Sequence
  peptide sequence
LAPYIP

SEQ ID NO: 13 - protein, 6 aa, Artificial Sequence
  peptide sequence
LAPYIG

(19) United States
(12) Patent Application Publication
(10) Pub. No.: US 2010/0240069 A1
(43) Pub. Date: Sep. 23, 2010

(54) Drug Discovery Assay for Modulators of HIF-Prolyl Hydroxylase Activity

(75) Inventors: John Alan Broadwater, Ridgefield, CT (US); Hnin Hnin Khine, Ridgefield, CT (US); Siqi Lin, Ridgefield, CT (US); Richard D. Schneiderman, Ridgefield, CT (US)

Correspondence Address: Michael P. Morris;Boehringer Ingelheim USA Corporation, 900 Ridgebury Road, Ridgefield, CT, 06877-0368, US

(73) Assignee: Boehringer Ingelheim International GmbH, Ingelheim (DE)

(21) Appl. No.: 12/406,691

(22) Filed: Mar. 18, 2009

(51) Int. Cl. G01N 33/573; C12Q 1/26

(52) U.S. Cl. 435/7.5; 435/25

(57) ABSTRACT

The present invention relates to a high throughput assay for determining HIF 1α prolyl hydroxylation.

### RELATED APPLICATIONS

[Not Applicable]

### FEDERALLY SPONSORED RESEARCH OR DEVELOPMENT

[Not Applicable]

### FIELD OF THE INVENTION

The present invention relates to assays for use in identification of modulators of hypoxia-inducible factors.

### BACKGROUND OF THE INVENTION

Hypoxia, physiologically the condition in which an animal's oxygen demand exceeds the supply, is known to have detrimental effects in various conditions. For example, regions of hypoxia are common in breast carcinoma where the rate of nutrient and oxygen consumption is insufficient to meet the metabolic demands of neoplastic cells. Other systemic, local, and intracellular homeostatic responses elicited by hypoxia include angiogenesis, erythropoiesis, neovascularization in ischemic myocardium, and glycolysis in cells cultured at reduced O₂ tension, vasomotor control, inflammation, tissue matrix metabolism, cell survival decisions, and tissue ischemia resulting from chronic hypoxia occurring from, for example, stroke, deep vein thrombosis, pulmonary embolus, and renal failure. Ischemic tissue is also found in tumors.

The adaptive responses to hypoxia either increase O₂ delivery or activate alternate metabolic pathways that do not require O₂. Hypoxia-inducible gene products that participate in these responses include erythropoietin (EPO), vascular endothelial growth factor (VEGF), and glycolytic enzymes. Hypoxia-inducible factor (HIF) is a transcription factor that plays a central role in cellular adaptation to hypoxia.

HIF is a heterodimeric transcription factor composed of two subunits. HIF-α and HIF-β. The alpha subunit is oxygen-dependent, whereas the beta subunitβ (also known as aryl hydrocarbon receptor nuclear translocator, ARNT) is constitutively expressed. While the alpha subunit is unique to each HIF, the beta subunit typically can dimerize with other proteins.

HIF plays a central role in cellular adaptation to hypoxia by transcriptionally upregulating target genes involved in angiogenesis, erythropoiesis, glycolysis, and the like. Renal disease pathology has been linked to hypoxia of tubulointerstitium. HIF, which is activated in kidney diseases, is thought to protect tubulointerstitium from the ischemia. The expression of HIF α-chains is post-transitionally regulated and hydroxylation at one or two of the conserved proline residues by Prolyl Hydroxylase (HPH) enzymes is an essential step in the oxygen-dependent recruitment of the von Hippel-Lindau gene product (pVHL), a recognition component of the E3 ubiquitin ligase complex, and degradation of HIF-α. It also has been shown that inhibition of PHD enzyme activity activates HIF irrespective of oxygenation status. Thus, PHD enzymes are good targets for use in screening pharmacological agents that will be useful in treating conditions resulting from a hypoxia-mediated response.

To permit the identification of modulators of HPH enzyme it would be ideal to have a high-throughput screening assay that is non-radioactive, robust, sensitive, and amenable to automation. While HPH assays have been reported, none of the currently available assays satisfy these assay criteria.

The complex reaction catalyzed by HIF-1α Prolyl Hydroxylase (HPH) has been assayed previously (McNeill et al. Bioorg. Med. Chem. Lett., 12, 1547-1550, 2002) by monitoring ¹⁴CO₂ production from ¹⁴C-α-ketoglutarate. However this assay format is problematic due to an uncoupling reaction that can occur in the absence of peptide substrate, the difficulty in measuring radioactive gas (¹⁴CO₂) in a high-throughput format, and due to the environmental and fiscal disadvantages of using radioactivity.

Oehme et al. (Anal. Biochem., 330, 74-80, 2004) have improved on this assay strategy by employing an ELISA format to detect the interaction of hydroxylated peptide and VHL protein. In the assay the VHL protein, in complex with its partners elongin B and elongin C (VBC), is tagged with thioredoxin and the biotinyl-peptide is immobilized in a 96-well streptavidin plate. VBC protein that remains bound to the plate during washing steps, via interaction with the hydroxylated peptide, is detected by addition of an anti-thioredoxin antibody and then a secondary antibody conjugated with horseradish peroxidase is added to permit colorimetric detection.

While the Oeheme et al. assay provides high-throughput capabilities, the colorimetric format is susceptible to interference -from compounds that may absorb light of a similar wavelength and is not sensitive enough to support large scale screening. Finally, Cho et al (Biochem. Biophysic. Res. Comm. 337, 275-280, 2005) have recently published a fluorescence polarization assay for HPH that would enable high-throughput screening without the need for radioactivity. In this format, HPH is used to convert a fluorescently labeled peptide substrate to its hydroxylated form. The binding of VBC protein to the hydroxylated peptide leads to an increase in the molecular rotation (due to its increased size) and this leads to an increase in the fluorescence polarization of the labeled peptide. However, the poor assay sensitivity requires impractically high amounts of the HPH enzyme as well as the VBC protein required to complete a high-throughput screening campaign.

Thus, there remains a need for a non-radioactive, robust, sensitive, and amenable to automation that can be used as a high-throughput screening assay in the identification of modulators of HPH enzyme. None of the currently available assays satisfy these assay criteria.

### BRIEF SUMMARY OF THE INVENTION

The present invention provides methods for determining hypoxia-inducible factor prolyl hydroxylation activity comprising:

a. incubating a mixture comprising a biotinylated proline-containing substrate and an HPH enzyme in a suitable buffer for a sufficient time to allow hydroxylation of the substrate;

b. quenching the reaction with a composition comprising a VBC complex attached to a GST (GST-VBC), streptavidin-labeled allophycocyanin and a fluorescently labeled anti-GST antibody for a time sufficient to allow the VBC complex to interact with the proline-containing substrate; and

c. determining the amount of interaction of the GST-VBC with the substrate.

In preferred embodiments, the HPH enzyme is selected from the group consisting of a HPH 1, HPH 2, and an HPH 3 enzyme. Preferably, the HPH enzyme is a mammalian HPH enzyme, however it the enzyme may be a non-mammalian ortholog of a mammalian HPH enzyme. Preferably, the HPH enzyme is a human HPH enzyme or in other embodiments, the HPH enzyme is a monkey HPH enzyme, such as a a rhesus monkey HPH enzyme.

In specific embodiments, the HPH enzyme comprises a sequence selected from the group consisting of SEQ ID NO:1; SEQ ID NO:2, SEQ ID NO:3; SEQ ID NO:4, SEQ ID NO: 5, and SEQ ID NO:6. In on particular assay, the HPH enzyme used is a rhesus monkey HPH having a sequence of SEQ ID NO:3.

In the methods of the invention the proline-containing substrate is a peptide comprising the sequence LXXLAP, wherein P in the substrate is hydroxylated by the HPH and X represents any amino acid. More particularly, the proline-containing substrate is a peptide comprising the sequence LAP*Y, wherein P* denotes the proline residue in the substrate that is hydroxylated by the HPH. In specific embodiments, the proline-containing substrate is a peptide comprising the sequence LAPYI, wherein P in the substrate is hydroxylated by the HPH. In other more specific embodiments, the proline-containing substrate is a peptide comprising the sequence of LAP*YIP or LAP*YIG, wherein P* in the substrate is hydroxylated by the HPH. Specific peptides that may serve as substrates in the assays described herein include but are not limited to a peptide comprising a sequence selected from the group consisting of DLDLEALAPYIPADDDFQL (SEQ ID NO:7), DLDLEMLAPYIPMDDDFQL(SEQ ID NO:8), DLDLEMLAPYIGMDDDFQL (SEQ ID NO:9), and or DLDLEALAPYIGADDDFQL (SEQ ID NO:10).

In particular aspects of the invention, the reaction mixture in the assay further comprises 2□-ketoglutarate as a substrate.

In specific embodiments, the fluorescently labeled anti-GST antibody comprises a fluorescent label selected from the group consisting of europium (Eu), dysprosium (Dy), lanthanum (La), gadolinium (Gd), cerium (Ce), samarium (Sm), yttrium (Y), neodymium (Nd), terbium (Tb), praseodymium (Pr), erbium (Er), thulium (Tm), ytterbium (Yb), scandium (Sc), promethium (Pm), holmium (Ho), and lutetium (Lu).

The detection step in the assays of the invention preferably comprises use of a time resolved homogeneous fluorescence assay. In preferred such assays, the fluorescently labeled anti-GST antibody is labeled with Europium. More particularly, in such an assay using an anti-GST antibody labeled with Europium, the detecting comprises detecting the signal at 665 nm and the signal at 620 nm, wherein the ratio of the signal at 665:620 nm proportional to the amount of peptide interaction with the GST-VBC.

In exemplary embodiments, the the assay is performed in the presence of a modulator of HPH activity.

The assay may be set up to perform the method as a high throughput screening assay in a multi-well plate format.

Another aspect of the invention details a method of screening for a modulator of HIF prolyl hydroxylation, comprising the steps of:

a. incubating a mixture comprising an isolated HIF-specific prolyl hydroxylase (HPH) protein and a peptide substrate of HPH hydroxylase enzymes, under conditions whereby a proline residue on the substrate is hydroxylated, and

b. quenching the reaction with a composition comprising a VBC complex attached to a GST (GST-VBC), streptavidin-labeled allophycocyanin and a fluorescently labeled anti-GST antibody for a time sufficient to allow the VBC complex to interact with the proline-containing substrate, and

c. detecting a resultant prolyl hydroxylation of the substrate using time resolved homogeneous fluorescence;

wherein the incubating step is performed in the presence and absence of candidate modulator of prolyl hydroxylation wherein an increase in the prolyl hydroxylation in the presence of the candidate modulator is indicative of the modulator being an enhancer of prolyl hydroxylation and a decrease in the prolyl hydroxylation in the presence of the candidate modulator is indicative of the modulator being an inhibitor of prolyl hydroxylation.

More particularly, such a screening method uses an HPH selected from the group consisting of a protein of SEQ ID NO:1, SEQ ID NO:2 and SEQ ID NO: 3.

Also taught herein is a method of determining HIF prolyl hydroxylation activity, comprising the steps of:

a. incubating a mixture comprising an isolated HIF-specific prolyl hydroxylase (HPH) protein from M. mulatta and a peptide substrate having a sequence of biotin-DLDLEMLAPYIPMDDDFQL, under conditions whereby a proline residue on the substrate is hydroxylated, and

b. quenching the reaction with a composition comprising a VBC complex attached to a GST (GST-VBC), streptavidin-labeled allophycocyanin and an europium-labeled anti-GST antibody for a time sufficient to allow the VBC complex to interact with the proline-containing substrate; and

c. detecting a resultant prolyl hydroxylation of the substrate using time resolved homogeneous fluorescence at an excitation wavelength of 330 nm; and emission at wavelengths 620 and 665 nm.

Preferably, the incubating step is performed in the presence and absence of candidate modulator of prolyl hydroxylation wherein an increase in the prolyl hydroxylation in the presence of the candidate modulator is indicative of the modulator being an enhancer of prolyl hydroxylation and a decrease in the prolyl hydroxylation in the presence of the candidate modulator is indicative of the modulator being an inhibitor of prolyl hydroxylation.

In a specific exemplary screening assay, the reaction mixture comprises a reaction buffer containing 20 mM MOPS (pH 6.5), 1.5 mM magnesium chloride, 5 mM potassium chloride. 0.1% bovine serum albumin, 1 mM TCEP, 10 □M ferrous ammonium sulfate, and 2 mM sodium ascorbate, the peptide substrate, □-ketoglutarate and, HPH enzyme.

These and other aspects of the present invention will become apparent upon reference to the following detailed description and attached drawings. All references disclosed herein are hereby incorporated by reference in their entirety as if each was incorporated individually.

### BRIEF DESCRIPTION OF SEVERAL VIEWS OF THE DRAWINGS

FIG. 1. Schematic description of the HPH assay used to identify inhibitors or stimulators of HPH.

FIG. 2. Representative data obtained with the assay exemplifying inhibition of HPH1 enzyme with succinate. The y-axis represents the Percent of Control and the x-axis the concentration of succinate (mM).

FIG. 3. Representative data obtained with the assay exemplifying inhibition of HPH2 enzyme with succinate. The y-axis represents the Percent of Control and the x-axis the concentration of succinate (mM).

FIG. 4. Representative data obtained with the assay exemplifying inhibition of HPH3 enzyme with succinate. The y-axis represents the Percent of Control and the x-axis the concentration of succinate (mM).

### DETAILED DESCRIPTION OF THE INVENTION

The hypoxia hypoxia-inducible factor 1 (HIF-1) is a ubiquitously expressed transcriptional master regulator of many genes regulating mammalian oxygen homeostasis. It has been shown to be involved in erythropoiesis, iron metabolism, angiogenesis, control of blood flow, glucose uptake and glycolysis, pH regulation, and cell-cycle control. Hydroxylation of HIF-1α at proline residues by HPH enzymes is an essential step in the oxygen-dependent degradation of HIF-1α. HPH enzymes are thus recognized as good targets for use in screening pharmacological agents that will be useful in treating conditions resulting from a hypoxia-mediated response. However, to date that assays available to screen for the activities of these enzymes are inadequate for use in high throughput format.

The present invention relates to a method that is readily amenable to a high throughput assay format that measures the activity of HPH proteins and is dependent upon VBC-recognition of a hydroxylated peptide substrate.

More specifically, the HPH enzyme assay is conducted in a multi-well plate format. In a suitable buffer, a peptide substrate for the HPH, and a substrate for a protein-based pull down assay, and HPH enzyme are incubated for a period of time and at a temperature sufficient to allow the hydroxylation of the proline-containing substrate to occur. After the incubation period (e.g., 45 min.) the reaction is quenched by the addition of a solution containing the detection reagents. In specific embodiments, the solution comprises EDTA (2.5 mM) and the detection reagents (GST-VBC, Streptavidin-allophycocyanin and Europium-labeled anti-GST Antibody). The detection method in this assay is time-resolved fluorescent assay in which the time-resolved fluorescent signal (excitation at 330 nm; emission at 620 and 665 nm) is read on a plate reader after 1 of incubation. The ratio of signal at 665 nm to 620 nm is used to calculate the amount of peptide interaction with GST-VBC as a measure of protein hydroxylation.

It should be understood that the person skilled in the art will be able to modify individual components of the assay and still be able to conduct an assay within the scope of the method. Further, the incubation times and buffer conditions also may be varied.

In an exemplary assay, the assay is conducted in a 96 or 384-well plate format. In the 384-well scale, the reaction (10 μl) contains buffer (20 mM MOPS (pH 6.5), 1.5 mM magnesium chloride, 5 mM potassium chloride, 0.1% bovine serum albumin, 1 mM TCEP, 10 μM ferrous ammonium sulfate, and 2 mM sodium ascorbate), substrate (2 μM α-ketoglutarate and 60 nM HIF-1αs peptide (biotin-DLDLEMLAPYIPMDDDFQL), enzyme (e.g. 8 nM of HPH3), and an inhibitor from a DMSO solution (1% DMSO final in the assay). After 45 min, the reaction is quenched by the addition of EDTA (2.5 mM) and the detection reagents (GST-VBC, Streptavidin-allophycocyanin and Europium-labeled anti-GST Antibody). The time-resolved fluorescent signal (excitation at 330 nm; emission at 620 and 665 nm) is read on a plate reader after 1 h of incubation. The ratio of signal at 665 nm to 620 nm is used to calculate the amount of peptide interaction with GST-VBC as a measure of protein hydroxylation.

The HPH enzyme used herein may be any HPH enzyme known to those of skill in the art. For example, Genbank Accession No. NP_{—}730908 depicts the HIF prolyl hydroxylase, isoform B from Drosophila melanogaster, NP_{—}730906 depicts the HIF prolyl hydroxylase, isoform C from Drosophila melanogaster, NP_{—}649525 depicts the HIF prolyl hydroxylase, isoform A from Drosophila melanogaster, Q9GZT9 depicts a sequence of HPH 2 from homo sapiens; Q96KS0 depicts a sequence of HPH 3 from homo sapiens, Q62630 depicts a sequence of HPH 3 from Rattus norvegicus, Q9H6Z9 depicts a sequence of HPH 3 from homo sapiens, Q91YE3 depicts a sequence of HPH 2 from Mus musculus, Q91YE2 depicts a sequence of HPH 1 from Mus musculus, Q91UZ4 depicts a sequence of HPH 3 from Mus musculus, P59722 depicts a sequence of HPH 2 of Rattus norvegicus.

Other sequences of HPH 1, HPH 2, and HPH 3 are known to those of skill in the art. Indeed, HPH sequences have been published for human, mouse, rat, dogs, orangutan and chimp. HPH genes were originally labeled EGLN (1, 2, 3) for Egg-Laying Mutant Nine (derived from C. elegans gene name) which the protein was referred to as prolyl hydroxylase domain (PHD 2, 1, 3) or HIF-Prolyl Hydroxylase (HPH 1, 2, 3). Additionally, while the names of the proteins were interchangeable, the gene identifier (i.e., the notation of “1” “2” and “3”) has not been consistently employed in the literature. For example HPH 1, 2, 3 equate with PHD 2, 1 and 3 respectively, and with EGLN 1, 2, and 3, respectively. However, in other instances the literature did not follow this convention. In specific embodiments, the HPH proteins used in the assays of the present invention have a sequence of human HPH 1, HPH 2 or HPH 3 as shown in SEQ ID NO:4, SEQ ID NO:5, and SEQ ID NO:6, respectively. More preferred are assays in which the sequence is of the HPH 1, HPH 2 or HPH 3 of Macaca mulatta as shown in SEQ ID NO:1, SEQ ID NO:2, and SEQ ID NO:3, respectively.

For production methods the HPH enzymes may be expressed in e.g., bacterial cells such that they could be purified by affinity chromatography as soluble proteins. In exemplary embodiments, heterologous expression of these proteins was performed by subcloning the genes into pvl1393 vectors with either N- or G-terminal Glutathione S-Transferase (GST) tags to aid in purification of the proteins. SF9 cells were co-transfected with BaculoGold™ Linearized Baculovirus DNA (Becton Dickinson) and the complementing pvl1393 transfer vector containing the gene of interest. To identify recombinant virions, a plaque purification was completed and viral amplifications from one isolated plaque were done in SF9 cells. After two viral amplifications, sufficient amounts of a high titered pure virus stock were generated for large scale protein production. Expression was verified using an anti-GST antibodies at each step of the viral amplification process.

While the full length sequences may be preferred for use in the assays described herein, it should be understood that the assays also may be performed using biologically active fragments of the sequences of SEQ ID NO:1, SEQ ID NO:2, and SEQ ID NO:3, SEQ ID NO:4, SEQ ID NO:5, or SEQ ID NO:6. Further it is contemplated that the assays may be performed with hybrids of human and Macaca mulatta sequences shown herein. The exact sequence identity of the HPH enzyme is not important to the assay as long as the HPH enzyme performs to hydroxylate a proline on a suitable substrate such as a proline a residue Pro-564 in HIF 1α.

The prolyl hydroxylation substrate for use in the assay described herein may be any substrate routinely used in determining HIF-1α hydroxylation by the HPH enzymes. Exemplary substrates are described in e.g., Huang et al (“Sequence Determinants in Hypoxia-inducible Factor-1α for Hydroxylation by the Prolyl Hydroxylases PHD1, PHD2, and PHD3” J. Biol. Chem., Vol. 277, No. 42, Issue of October 18, pp. 39792-39800, 2002). As noted therein, HPH enzymes hydroxylate specific prolines in HIF subunits in the context of a strongly conserved LXXLAP sequence motif (where X indicates any amino acid and P indicates the hydroxyl acceptor proline). IT is noted that in substrates for an HPH assay that comprise a sequence of LXXLAP mutations can be readily tolerated at the -5, -2, and -1 positions (relative to proline) of the LXXLAP motif.

Specific exemplary peptides that can serve as substrates include peptides of that comprise the sequence LAPY. The peptides may be of any length and any sequence as long as the sequence and length of the peptide renders them readily amenable to prolyl hydroxylation. As noted in Huang et al. supra the only obligatory residue for proline hydroxylation in HIF-b 1α is the hydroxyl acceptor proline itself. Thus, any peptide based on the proline hydroxylation motif of HIF 1α which retains the LXXLAP motif but has a longer and different sequence in other aspects of the peptide may be used as a substrate for the assay. For example, it has been described that a double Met to Ala substitution in a human HIF 1α ODD domain provide an equivalent HDH substrate with less oxidative reactivity and hence, improved compatibility with mass spectroscopy-based analysis. In general, preferred substrates comprise the peptide LAPY, more preferably LAPYI (SEQ ID NO:11), more preferably LAPYI, wherein the I is coupled to an additional residue, preferably P or G. Thus, peptides that comprise the sequence LAPYIP (SEQ ID NO:12) or LAPYIG (SEQ ID NO:13) are specifically contemplated to be useful substrates for the enzymes described herein Exemplary peptides that may be used include for example, DLDLEMLAPYIPMDDDFQL (SEQ ID NO:7), DLDLEMLAPYIGMDDDFQL (SEQ ID NO:8), DLDLEALAPYIPADDDFQL (SEQ ID NO:9) or DLDLEALAPYIGADDDFQL (SEQ ID NO:10).

In preferred embodiments, the substrate is biotinylated.

The screening assays ideally will be set up to assay for candidate substances that modulate the resultant prolyl hydroxylation, wherein an agent-biased prolyl hydroxylation is detected. Candidate agents encompass numerous chemical classes, though typically they are organic compounds; preferably small organic compounds and are obtained from a wide variety of sources including libraries of synthetic or natural compounds. A variety of other reagents may also be included in the mixture. These include reagents like cofactors (e.g. Fe(II)), cosubstrates (e.g. dioxygen), salts, buffers, neutral proteins, e.g. albumin, detergents, protease inhibitors, antimicrobial agents, etc.

The candidate modulators can be any small molecule compound, or a biological entity, such as a protein, sugar, nucleic acid or lipid. Typically, test compounds will be small chemical molecules and peptides. Essentially, any chemical compound can be used as a potential HPH agonist or antagonist, although most often compounds that can be dissolved in aqueous or organic (especially DMSO-based) solutions are used. In general, assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

Preparation and screening of combinatorial chemical libraries is well known to those of skill in the art. Such combinatorial chemical libraries include, but are not limited to, peptide libraries (see, e.g., U.S. Pat. No. 5,010,175). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides, vinylogous polypeptides, nonpeptidal peptidomimetics with glucose scaffolding, analogous organic syntheses of small compound libraries, oligocarbamates, and/or peptidyl phosphonates, nucleic acid, peptide nucleic acid libraries (see, e.g. U.S. Pat. No. 5,539,083), antibody libraries (see, e.g., PCT/US96/10287), carbohydrate libraries (see, e.g., U.S. Pat. No. 5,593,853), small organic molecule libraries (see, e.g., benzodiazepines; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 NPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Using lead compounds to help develop improved compounds is know as “rational drug design” and includes not only comparisons with know inhibitors and activators, but predictions relating to the structure of target molecules. The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches. It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

The assay format used herein for the high throughput screening methods of the present invention is cell free high throughput screening assay format. In this cell-free assay format, substantially purified HPH proteins (either isolated from natural sources or recombinantly produced), partially purified or crude cellular extracts will be used as the enzymes for catalyzing the proline hydroxylation. The screening assays of the invention detect the HPH activity using a homogenous time resolved fluorescence (HIT-Fret) assays used to determine the activity of a given compound to serve as a modulator of prolyl hydroxylation.

This assay is based on VBC-recognition of a hydroxylated peptide. In this assay, the VHL protein, in complex with its partners, elongin B and elongin C (VBC), is conjugated to a Glutathione-S transferase enzyme (GST). This may be readily achieved using recombinant methods for producing expression of the entire GST-VBC sequence. Alternatively, GST protein, which is well known to those of skill in the art is physically conjugated to a VBC complex using covalent bonding through a linker. In exemplary embodiments, heterologous expression of the VBC complex proteins can be performed by subcloning the genes from these proteins into appropriate vectors with either N- or C-terminal Glutathione S-Transferase (GST). In exemplary embodiments, the VBC protein complex was generated by co-expression of Glutathione S-Transferase-VHL (von Hippel Lindau), elongin B and elongin C in Escherichia coli followed by purification of the complex using glutathione affinity resin and size-exclusion chromatography.

For production methods the VBC complex may be expressed in e.g., bacterial cells such that they could be purified by affinity chromatography as soluble proteins. In exemplary embodiments, heterologous expression of these proteins was performed by subcloning the elongin B and C genes into a pCDF vector (Novagen) and the VHL gene into a pDEST15 vector with an N-terminal Glutathione S-Transferase (GST) tag and the resultant vectors were transformed into Escherichia coli BL21 cells (Novagen) as described in the manufacturer's instructions. Expression was verified using an anti-GST antibody and the protein complex was purified using glutathione affinity chromatography and size-exclusion chromatography (GE Healthcare).

In the assay, the biotinylated substrate is captured by the Streptavidin which is attached to allophycocyanin. The VBC interacts with the substrate and the interaction is recognized through the aid of an anti GST antibody which is labeled with europium. When the VBC is bound to the substrate, the europium is brought into proximity with the allophycocyanin. Bringing the europium and allophycocyanin into close proximity generates a signal. The time-resolved fluorescent signal (excitation at 330 nm; emission at 620 and 665 nm) is read on a plate reader after 1 h of incubation. The ratio of signal at 665 nm to 620 nm is used to calculate the amount of peptide interaction with GST-VBC as a measure of protein hydroxylation.

In the present invention the interaction of europium and allophycocyanin is used as an exemplary fluorescent signal generation couple. Those skilled in the art will recognize that other fluorescent signals may be generated by labeling with dysprosium (Dy), lanthanum (La), gadolinium (Gd), cerium (Ce), samarium (Sm), yttrium (Y), neodymium (Nd), terbium (Tb), praseodymium (Pr), erbium (Er), thulium (Tm), ytterbium (Yb), scandium (Sc), promethium (Pm), holmium (Ho), and lutetium (Lu) and the like.

The activity of recombinant HPH protein may be stimulated in the assays by the addition of ascorbate, 2-oxoglutarate and FeSO4. Thus, screening assays designed to use the HPH compositions of the present invention may preferably include such reagents. As it is known that CoCl2 induces the hypoxic response pathway by stabilizing HIF under normoxic conditions, possibly by competing with Fe2+ for occupancy within the active site of HPH, the screening assays may be performed in the presence and absence of CoCl2 in order to assess the likelihood that the candidate substance being tested will be able to overcome a hypoxic effect.

The screening assay may measure a candidate agent's ability to specifically bind to or modulate activity of an HPH polypeptide, a fusion protein thereof, or to cells or membranes bearing the polypeptide or fusion protein. The HPH polypeptide can be full length or a fragment thereof that retains functional HPH activity. The HPH polypeptide may be fused to another polypeptide, such as a peptide tag for detection or anchoring, or to another tag.

Once the screening assay has identified an appropriate candidate modulator it may be further tested and optimized for its activity in the HPH assays of the invention and in animal models for further testing of the use of the same in various indications.

### EXAMPLE

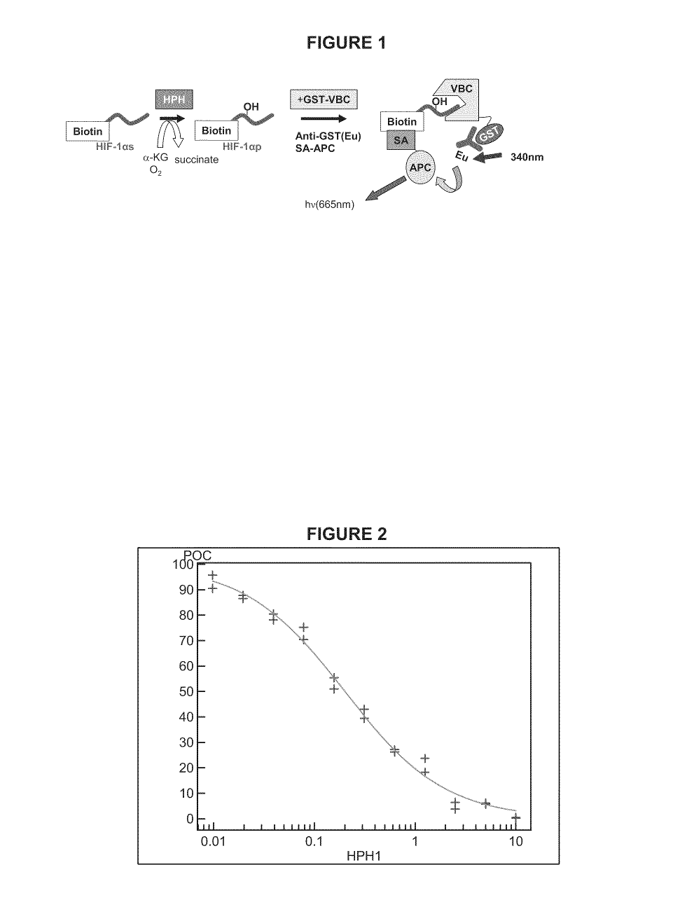

An exemplary assay schematic of the present invention is shown in FIG. 1. The VBC protein is fused to the protein glutathione S-transferase (GST) and a europium-labeled anti-GST antibody is bound to GST while a streptavidin-allophycocyanin (SA-APC) conjugate is used to bind the biotinylated-peptide substrate. The result of this complex assemble of proteins is that the fluorescent europium is brought close enough to the fluorescent APC molecule to permit Time-Resolved Fluorescent Resonant Energy Transfer (TR-FRET) at a unique wavelength (665 nm) The homogeneous nature of this assay provides for a faster and more reproducible assay than an ELISA format and one that is amenable to high-throughput screening.

In the present exemplary assay, recombinant rhesus HPH(1-3) enzyme, derived from an SF9 expression system were used (i.e., proteins having the sequence of SEQ ID NO:1, SEQ ID NO:2 or SEQ ID NO:3). However, it should be understood that in principal any enzyme capable of hydroxylating the Pro-containing peptide could be substituted for these exemplary HPH proteins. The VBC protein complex was generated by co-expression of Glutathione S-Transferase-VHL (von Hippel Lindau), elongin B and elongin C in Escherichia coli followed by purification of the complex using glutathione affinity resin and size-exclusion chromatography.

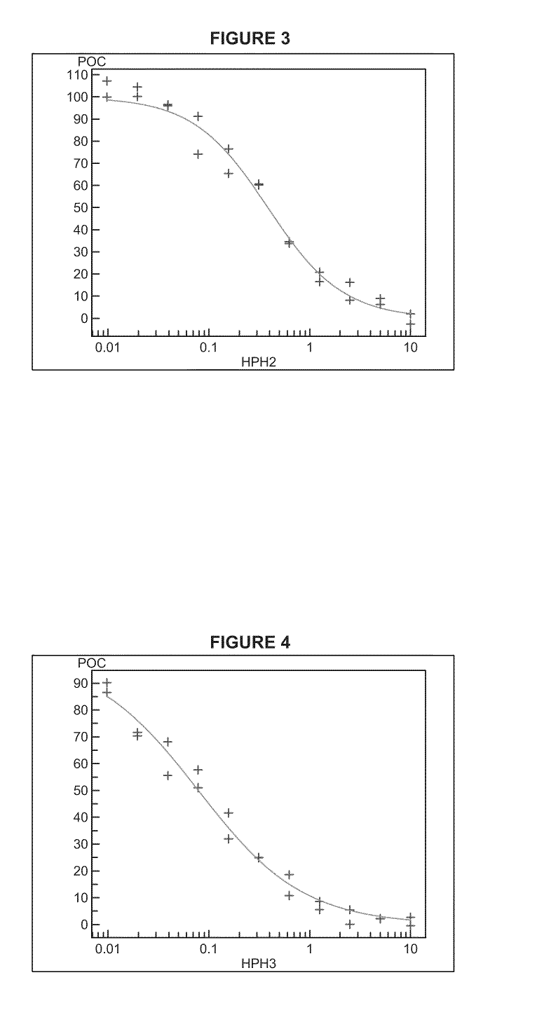

Exemplary results of the inhibition of enzyme activity are shown in FIGS. 2-4, and Table 1.

TABLE 1
Inhibitory activity of known HPH inhibitors against rhesus HPH.

| | IC₅₀ (μM) | | |
|---|---|---|---|
| | HPH1 | HPH2 | HPH3 |
| Succinate | 200 | 380 | 80 |
| Hydralazine | 400 | 320 | 54 |
| N-oxalylglycine | nd | nd | 0.66 |

These data show that the recombinant rhesus HPH protein was catalytically active and exhibited a similar degree of inhibitor binding as the human counterparts. Similarly, it was found that the addition of ascorbate and ferrous iron increased the activity of the enzyme whereas CoCl₂ inhibited enzyme activity. The specific activity of the recombinant enzymes was determined to be in the range of 3-10 nmol/min/mg which is in the range of previously reported activities from the human HPH enzymes (Tuckerman J. R., Zhao, Y., Hewitson, K. S., Tian, Y.-M., Pugh, C. W., Ratcliffe, P. J., Mole, D. R. (2004) FEBS Lett. 576:145-150 (2004).

1. A method for determining hypoxia-inducible factor prolyl hydroxylation activity comprising:
a. incubating a mixture comprising a biotinylated proline-containing substrate and an HPH enzyme in a suitable buffer for a sufficient time to allow hydroxylation of said substrate;
b. quenching said reaction with a composition comprising a VBC complex attached to a GST (GST-VBC), streptavidin-labeled allophycocyanin and a fluorescently labeled anti-GST antibody for a time sufficient to allow the VBC complex to interact with said proline-containing substrate; and
c. determining the amount of interaction of said GST-VBC with said substrate.

2. The method of claim 19 wherein said HPH enzyme is selected from the group consisting of a HPH 1, HPH 2, and an HPH 3 enzyme.

3. The method of claim of claim 2, wherein said HPH enzyme is a mammalian HPH enzyme.

4. The method of claim of claim 3, wherein said HPH enzyme is a human HPH enzyme.

5. The method of claim of claim 3, wherein said HPH enzyme is a monkey HPH enzyme.

6. The method of claim of claim 3, wherein said HPH enzyme is a rhesus monkey HPH enzyme.

7. The method of claim 3, wherein said HPH enzyme has a sequence selected from the group consisting of SEQ ID NO:1; SEQ ID NO:2, SEQ ID NOS:3; SEQ ID NO:4, SEQ ID NO: 5, and SEQ ID NO:6.

8. The method of claim 3, wherein said HPH enzyme is rhesus monkey HPH having a sequence of SEQ ID NO:3.

9. The method of claim 1, wherein said proline-containing substrate is a peptide comprising the sequence LXXLAP, wherein P in said substrate is hydroxylated by the HPH and X represents any amino acid.

10. The method of claim 1, wherein said proline-containing substrate is a peptide comprising the sequence LAPY, wherein P in said substrate is hydroxylated by the HPH.

11. The method of claim 19 wherein said proline-containing substrate is a peptide comprising the sequence LAPYI, wherein P in said substrate is hydroxylated by the HPH.

12. The method of claim 1, wherein said proline-containing substrate is a peptide comprising the sequence of LAPYIP or LAPYIG, wherein P in said substrate is hydroxylated by the HPH.

13. The method of claim 1, wherein said proline-containing substrate is a peptide comprising a sequence selected from the group consisting of DLDLEALAPYIPADDDFQL, DLDLEMLAPYIPMDDDFQL, DLDLEMLAPYIGMDDDFQL, and or DLDLEALAPYIGADDDFQL (SEQ ID NO______).

14. The method of claim 1, wherein the reaction mixture in step a further comprises 2α-ketoglutarate as a substrate.

15. The method of claim 1 wherein said fluorescently labeled anti-GST antibody comprises a fluorescent label selected from the group consisting of europium (Eu), dysprosium (Dy), lanthanum (La), gadolinium (Gd), cerium (Ce), samarium (Sm), yttrium (Y), neodymium (Nd), terbium (Tb), praseodymium (Pr), erbium (Er), thulium (Tm), ytterbium (Yb), scandium (Sc), promethium (Pm), holmium (Ho), and lutetium (Lu).

16. The method of claim 1, wherein the detecting comprises a time resolved homogeneous fluorescence assay.

17. The method of claim 16, wherein said fluorescently labeled anti-GST antibody is labeled with Europium.

18. The method of claim 17, wherein said detecting comprises detecting the signal at 665 nm and the signal at 620 nm, wherein the ratio of the signal at 665:620 nm proportional to the amount of peptide interaction with said GST-VBC.

19. The method of claim 1, wherein said assay is performed in the presence of a modulator of HPH activity.

20. The method of claim 1, wherein said method is a high throughput screening assay in a multi-well plate format.

21. A method of screening for a modulator of HIF prolyl hydroxylation., comprising the steps of:
a. incubating a mixture comprising an isolated HIF-specific prolyl hydroxylase (HPH) protein and a peptide substrate of HPH hydroxylase enzymes, under conditions whereby a proline residue on said substrate is hydroxylated, and
b. quenching said reaction with a composition comprising a VBC complex attached to a GST (GST-VBC), streptavidin-labeled allophycocyanin and a fluorescently labeled anti-GST antibody for a time sufficient to allow the VBC complex to interact with said proline-containing substrate; and
c. detecting a resultant prolyl hydroxylation of the substrate using time resolved homogeneous fluorescence;
wherein the incubating step is performed in the presence and absence of candidate modulator of prolyl hydroxylation wherein an increase in the prolyl hydroxylation in the presence of said candidate modulator is indicative of said modulator being an enhancer of prolyl hydroxylation and a decrease in the prolyl hydroxylation in the presence of said candidate modulator is indicative of said modulator being an inhibitor of prolyl hydroxylation.

22. The method of claim 21, wherein said HPH is selected from the group consisting of a protein of SEQ ID NO:1, SEQ ID NO:2 and SEQ ID NO: 3.

23. A method of determining HIF prolyl hydroxylation activity, comprising the steps of:
a. incubating a mixture comprising an isolated HIF-specific prolyl hydroxylase (HPH) protein from M. mulatta and a peptide substrate having a sequence of biotin-DLDLEMLAPYIPMDDDFQL, under conditions whereby a proline residue on said substrate is hydroxylated, and
b. quenching said reaction with a composition comprising a VBC complex attached to a GST (GST-VBC), streptavidin-labeled allophycocyanin and an europium-labeled anti-GST antibody for a time sufficient to allow the VBC complex to interact with said proline-containing substrate; and
c. detecting a resultant prolyl hydroxylation of the substrate using time resolved homogeneous fluorescence at an excitation wavelength of 330 nm; and emission at wavelengths 620 and 665 nm.

24. The method of claim 23, wherein said incubating step is performed in the presence and absence of candidate modulator of prolyl hydroxylation wherein an increase in the prolyl hydroxylation in the presence of said candidate modulator is indicative of said modulator being an enhancer of prolyl hydroxylation and a decrease in the prolyl hydroxylation in the presence of said candidate modulator is indicative of said modulator being an inhibitor of prolyl hydroxylation.

25. The method of claim 23 wherein said reaction mixture comprises a reaction buffer containing 20 mM MOPS (pH 6.5), 1.5 mM magnesium chloride, 5 mM potassium chloride, 0.1% bovine serum albumin, 1 mM TCEP, 10 μM ferrous ammonium sulfate, and 2 mM sodium ascorbate, said peptide substrate, α-ketoglutarate and, HPH enzyme.
